(19) United States
(12) Patent Application Publication
(10) Pub. No.: US 2022/0362267 A1
(43) Pub. Date: Nov. 17, 2022

(54) CAPACITATIVE CALCIUM ENTRY INHIBITORS

(71) Applicants: UNIVERSITÉ PARIS-SACLAY, GIF SUR YVETTE (FR); INSERM (INSTITUT NATIONAL DE LA SANTE ET DE LA RECHERCHE MEDICALE), PARIS (FR); SORBONNE UNIVERSITÉ, PARIS (FR); CENTRE NATIONAL DE LA RECHERCHE SCIENTIFIQUE, PARIS (FR); UNIVERSITÉ DE BORDEAUX, BORDEAUX (FR); BORDEAUX INP, TALENCE (FR)

(72) Inventors: Olivier DELLIS, PARIS (FR); Mathieu PUCHEAULT, CAMBLANES-ET-MEYNAC (FR); Camille LE GUILCHER, PARIS (FR)

(21) Appl. No.: 17/623,477

(22) PCT Filed: Jun. 26, 2020
(86) PCT No.: PCT/FR2020/051121

§ 371 Date: Dec. 28, 2021

(30) Foreign Application Priority Data
Jun. 28, 2019 (FR) 1907184

(51) Int. Cl. A61K 31/69

(52) U.S. Cl. CPC A61K 31/69

(57) ABSTRACT

The invention relates to Orai1-dependent selective capacitative calcium entry inhibitors as well as to the therapeutic use thereof.

The present invention relates to Orai1-dependent selective capacitive calcium entry inhibitors and to the therapeutic use thereof.

The control of calcium homeostasis is a complex process that is essential at the cellular level, insofar as an increase in intracellular calcium ion concentration is involved in the regulation of numerous functions, including contraction, differentiation and proliferation. This increase is due to a release of calcium ions from internal stores, mainly from the endoplasmic reticulum (ER), and to an influx of calcium ions from the extracellular medium.

Calcium channels referred to as SOCCs (Store-Operated Calcium Channels) represent the main route of calcium influx into immune cells, including leukocytes (they are also known as “CRAC” (Calcium Release Activated Calcium) channels in lymphocytes and mast cells only). Calcium influx is controlled by two proteins, STIM1 and Orai1. STIM1 is a protein located in the ER membrane, which acts as a calcium sensor present in the ER lumen. Orai1 is a transmembrane protein present in the plasma membrane of leukocytes. A decrease in calcium concentration in the stores of the ER leads to an oligomerization of STIM1, which under these conditions interacts with the Orai1 proteins. This interaction then allows the Orai1 channels to open, leading to a massive influx of calcium ions from the extracellular medium. This calcium entry phenomenon controlled by the calcium concentration in the ER is called SOCE (store-operated calcium entry). Other channels, such as the 2 orthologs Orai2 and Orai3 and certain channels of the TRP family, form Orai1-independent SOCEs in other cell types. They can also serve as Orai1-associated proteins and modify the kinetic and pharmacological properties of Orai1 channels, but the Orai1 proteins remain those which form the pore of the channel

Orai1 was identified following the discovery of a point mutation in its sequence, resulting in a nonfunctional calcium channel in leukocyte cells, and in a severe combined immunodeficiency. The channels formed by Orai1 therefore play a crucial role in leukocyte functions and demonstrate that leukocyte SOCE is directly dependent on the presence and functionality of Orai1.

Compounds which inhibit Orai1-dependent CRAC channels, and therefore Orai1-dependent SOCE (hereinafter referred to as “Orai1-SOCE”), could therefore be advantageously used to control immune cell activation. 2-Aminoethoxydiphenyl borate (2-APB) has been described as an Orai1-SOCE modulator; however, its activity is dependent on the dose used. Between 1 and 5 μM, 2-APB potentiates Orai1-SOCE in leukocytes, but inhibits it at a concentration greater than 30 μM (Djillani et al., Biochim Biophys Acta 2014, 1843, 2341-7; Prakriya et al., J Physiol 2001, 536, 3-19). Moreover, it has been reported that 2-APB inhibits the inositol 1,4,5-triphosphate receptor (or IP3R; Ma et al., J Biol Chem 2002, 277, 6915-22; Bootman et al., FASEB J 2002, 16, 1145-50) and that it also inhibits ER calcium pumps (or SERCAs) (Missiaen et al., Cell Calcium 2001, 29, 111-6; Peppiatt et al., Cell Calcium 2003, 34, 97-108). Inositol 1,4,5-triphosphate (or IP3) is a secondary messenger, the synthesis of which is induced after stimulation of a cell by extracellular ligands such as during the interaction of the T lymphocyte with an antigen-presenting cell. IP3 is therefore capable of in turn inducing a release of calcium ions from the ER, after binding to the IP3R present on the cytoplasmic face of the ER. 2-APB is therefore a complex compound with no specificity, since, depending on the concentration at which it is used, it modulates, in a manner that is difficult to control, the activity of several elements having an impact on cellular calcium homeostasis.

Other SOCE inhibitors have been proposed, in particular the tandem 2-APB derivatives DPB162-AE and DPB163-AE (Goto et al., Cell Calcium 2010, 47, 1-10). However, at low concentration, the DPB163-AE compound activates SOCE (Goto et al., Cell Calcium 2010, 47, 1-10), whereas the DPB162-AE compound also affects the content of endoplasmic calcium reserves in various cell types (Bittremieux et al., Cell Calcium 2017, 62, 60-70).

Moreover, Hofer et al. (Bioorganic & Medicinal Chemistry 21 (2013) 3202-3213) have also developed 2-APB derivatives which are TRPV6-dependent calcium transport inhibitors. This study was carried out in HEK293T cells, which lack Orai1-SOCE. The article by Hofer et al. does not therefore provide any teaching for the synthesis of Orai1-SOCE inhibitors.

One of the objects of the present invention is to provide Orai1-SOCE inhibitor compounds which are selective at low dose, thus making possible their use as a medicament. It is in this context that the inventors have been able to demonstrate that a specific class of compounds, defined below, makes it possible to selectively inhibit Orai1-SOCE at low dose, without having an impact on IP3R or SERCA.

Thus, a subject of the invention is a compound of formula (I) or a pharmaceutically acceptable salt thereof, for use in the modulation of Orai1-dependent SOCE:

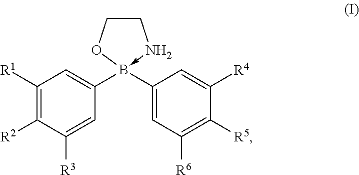

wherein R1, R2, R3, R4, R5 and R6 are groups, which may be identical or different, selected from a hydrogen atom, a halogen atom, a C₁-C₆ alkyl group, a C₁-C₆ alkenyl group, a C₁-C₆ alkyloxy group, a C₁-C₆ alkylthio group, a C₆-C₁₄ aryl group and a heteroaryl group comprising 5 to 14 ring members;
with the proviso that, when two of the R1, R2 and R3 groups represent a hydrogen, then the group among the R1, R2 and R3 which is not a hydrogen is selected from a C₃-C₆ alkyl group, a C₃-C₆ alkenyl group, a C₃-C₆ alkyloxy group, a C₃-C₆ alkylthio group, a C₆-C₁₄ aryl group and a heteroaryl group comprising 5 to 14 ring members;
with the proviso that, when two of the R4, R5 and R6 groups represent a hydrogen, then the group among the R4, R5 and R6 which is not a hydrogen is selected from a C₃-C₆ alkyl group, a C₃-C₆ alkenyl group, a C₃-C₆ alkyloxy group, a C₃-C₆ alkylthio group, a C₆-C₁₄ aryl group and a heteroaryl group comprising 5 to 14 ring members;
it being possible for the R1 and R2 groups to form, with the carbon atoms to which they are bound, a ring comprising 5 to 7 ring members;
it being possible for the R4 and R5 groups to form, with the carbon atoms to which they are bound, a ring comprising 5 to 7 ring members; and
said aryl or heteroaryl groups being optionally substituted with one or more substituents selected from halogen atoms, C₁-C₆ alkyl groups, C₂-C₆ alkenyl groups, C₁-C₆ alkyloxy groups, C₁-C₆ alkylthio groups, C₂-C₆ ketone groups, C₁-C₆ ester groups, C₁-C₆ aldehyde groups, —NH2, —CN, -CF3, C₁-C₆ amine groups and C₁-C₆ amine groups mono- or disubstituted with a C₁-C₆ alkyl group.

The invention also relates to a compound of formula (I), for use as a medicament.

The term “halogen” denotes a fluorine, chlorine, bromine or iodine atom.

The term “alkyl” denotes a linear or branched, saturated aliphatic group. The expression “C₁-C₆ alkyl” can more particularly denote a methyl, ethyl, propyl, isopropyl, n-butyl, isobutyl, sec-butyl, tert-butyl, pentyl or hexyl group.

The term “alkene” denotes a linear or branched aliphatic group comprising at least one carbon-carbon double bond.

The term “alkyloxy” denotes an —O-alkyl group where the alkyl group is as defined above. The expression “C₁-C₆ alkyloxy” can more particularly denote a methoxy, ethoxy, propyloxy, isopropyloxy, n-butyloxy, isobutyloxy, tert-butyloxy, sec-butyloxy, pentyloxy or hexyloxy group.

The term “alkylthio” denotes an —S-(alkyl) group, where the alkyl group is as defined above. The expression “C₁-C₆ alkylthio” can more particularly denote a methylthio, ethylthio, propylthio, isopropylthio, n-butylthio, isobutylthio, tert-butylthio, sec-butylthio, pentylthio or hexylthio group.

The term “aryl” denotes a monocyclic or polycyclic hydrocarbon-based aromatic group. More particularly, an aryl may denote a phenyl, a biphenyl or a naphthyl, and preferably a phenyl.

The term “heteroaryl” denotes a monocyclic or polycyclic aromatic group comprising at least one heteroatom such as nitrogen, oxygen or sulfur. More particularly, a heteroaryl can denote a pyridinyl, thiazolyl, thiophenyl, furanyl, pyrrolyl, imidazolyl, triazolyl, tetrazolyl, benzofuranyl, thianaphthalenyl, indolyl, indolinyl, quinolinyl, isoquinolinyl, benzimidazolyl, triazinyl, thianthrenyl, isobenzofuranyl, phenoxanthinyl, isothiazolyl, isoxazolyl, pyrazinyl, pyridazinyl, indolizinyl, isoindolyl, indazolyl, purinyl, phthalazinyl, naphthyridinyl, quinoxalinyl, quinazolinyl, cinnolinyl, pteridinyl, carbazolyl, β-carbolinyl, phenanthridinyl, acridinyl, pyrimidinyl, phenanthrolinyl, phenazinyl, phenothiazinyl, furazanyl, phenoxazinyl, benzotriazolyl, benzoxazolyl, benzisoxazolyl, oxindolyl, benzothienyl, benzothiazolyl, s-triazinyl, oxazolyl or thiofuranyl.

According to one particular embodiment, one group among R1, R2 and R3 represents a hydrogen atom. According to one variant of this embodiment, one group among R1, R2 and R3 represents a hydrogen atom and the other two groups from R1, R2 and R3 independently represent a halogen atom, a C₁-C₆ alkyl group, a C₁-C₆ alkenyl group, a C₁-C₆ alkyloxy group, a C₁-C₆ alkylthio group, a C₆-C₁₄ aryl group or a heteroaryl group comprising 5 to 14 ring members. According to one particular embodiment, R3 is a hydrogen atom and R1 and R2, which may be identical or different, represent a halogen atom, a C₁-C₆ alkyl group, a C₁-C₆ alkenyl group or a C₁-C₆ alkyloxy group or a C₁-C₆ alkylthio group. According to one particular embodiment, R3 is a hydrogen atom and R1 and R2, which may be identical or different, represent a halogen atom, a C₁-C₆ alkyl group, a C₁-C₆ alkenyl group or a C₁-C₆ alkyloxy group, or a C₁-C₆ alkylthio group. According to another embodiment, R3 is a hydrogen atom and R1 and R2, which may be identical or different, represent a halogen atom, a C₁-C₆ alkyl group, a C₁-C₆ alkenyl group or a C₁-C₆ alkyloxy group. Even more particularly, R3 may be a hydrogen atom and R1 and R2, which may be identical or different, can represent a halogen atom, a C₁-C₆ alkyl group or a C₁-C₆ alkyloxy group. According to another particular embodiment, R3 is a hydrogen atom and R1 and R2, which may be identical or different, represent a halogen atom or a C₁-C₆ alkyl group. According to one preferred embodiment, R3 represents a hydrogen atom, R1 represents a halogen atom or a C₁-C₆ alkyl group, and R2 represents a halogen atom or a C₁-C₆ alkyl group. According to one implementation variant, R3 represents a hydrogen atom, R1 represents a halogen atom, and R2 represents a C₁-C₆ alkyl group. According to another variant, R3 represents a hydrogen atom, R1 represents a C₁-C₆ alkyl group, and R2 represents a halogen atom. According to yet another variant, R3 represents a hydrogen atom, R1 represents a C₁-C₆ alkyl group, in particular methyl, and R2 represents a C₁-C₆ alkyl group, in particular methyl. More particularly, R3 represents a hydrogen atom, R1 represents a halogen atom, in particular chlorine, and R2 represents a methyl group. In another particular embodiment, R3 represents a hydrogen atom, R1 represents a methyl group and R2 represents a halogen atom, in particular a chlorine.

According to one particular embodiment, R2 is a hydrogen atom and R1 and R3, which may be identical or different, represent a halogen atom, a C₁-C₆ alkyl group, a C₁-C₆ alkenyl group, a C₁-C₆ alkyloxy group or a C₁-C₆ alkylthio group. According to another embodiment, R2 is a hydrogen atom and R1 and R3, which may be identical or different, represent a halogen atom, a C₁-C₆ alkyl group, a C₁-C₆ alkenyl group or a C₁-C₆ alkyloxy group. Even more particularly, R2 can be a hydrogen atom and R1 and R3, which may be identical or different, can represent a halogen atom, a C₁-C₆ alkyl group or a C₁-C₆ alkyloxy group. According to another particular embodiment, R2 is a hydrogen atom and R1 and R3, which may be identical or different, represent a halogen atom or a C₁-C₆ alkyl group. According to one preferred embodiment, R2 represents a hydrogen atom, R1 represents a halogen atom or a C₁-C₆ alkyl group, and R3 represents a halogen atom or a C₁-C₆ alkyl group. According to one implementation variant, R2 represents a hydrogen atom, R1 represents a halogen atom, and R3 represents a C₁-C₆ alkyl group. According to another variant, R2 represents a hydrogen atom, R1 represents a C₁-C₆ alkyl group, and R3 represents a halogen atom. According to yet another variant, R2 represents a hydrogen atom, R1 represents a C₁-C₆ alkyl group, in particular methyl, and R3 represents a C₁-C₆ alkyl group, in particular methyl. More particularly, R2 represents a hydrogen atom, R1 represents a halogen atom, in particular chlorine, and R3 represents a methyl group. In another particular embodiment, R2 represents a hydrogen atom, R1 represents a methyl group and R3 represents a halogen atom, in particular chlorine.

According to another particular embodiment, one group from R4, R5 and R6 represents a hydrogen atom. According to one variant of this embodiment, one group from R4, R5 and R6 represents a hydrogen atom and the other two groups from R4, R5 and R6 independently represent a halogen atom, a C₁-C₆ alkyl group, a C₁-C₆ alkenyl group, a C₁-C₆ alkyloxy group, a C₁-C₆ alkylthio group, a C₆-C₁₄ aryl group or a heteroaryl group comprising 5 to 14 ring members. According to one particular embodiment, R6 is a hydrogen atom and R4 and R5, which may be identical or different, represent a halogen atom, a C₁-C₆ alkyl group, a C₁-C₆ alkenyl group or a C₁-C₆ alkyloxy group or a C₁-C₆ alkylthio group. According to another embodiment, R6 is a hydrogen atom and R4 and R5, which may be identical or different, represent a halogen atom, a C₁-C₆ alkyl group, a C₁-C₆ alkenyl group or a C₁-C₆ alkyloxy group. Even more particularly, R6 may be a hydrogen atom and R4 and R5, which may be identical or different, can represent a halogen atom, a C₁-C₆ alkyl group or a C₁-C₆ alkyloxy group. According to another particular embodiment, R6 is a hydrogen atom and R4 and R5, which may be identical or different, represent a halogen atom or a C₁-C₆ alkyl group. According to one preferred embodiment, R6 represents a hydrogen atom, R4 represents a halogen atom or a C₁-C₆ alkyl group, and R5 represents a halogen atom or a C₁-C₆ alkyl group. According to one implementation variant, R6 represents a hydrogen atom, R4 represents a halogen atom, and R5 represents a C₁-C₆ alkyl group. According to another variant, R6 represents a hydrogen atom, R4 represents a C₁-C₆ alkyl group, and R5 represents a halogen atom. According to yet another variant, R6 represents a hydrogen atom, R4 represents a C₁-C₆ alkyl group, in particular methyl, and R5 represents a C₁-C₆ alkyl group, in particular methyl. More particularly, R6 represents a hydrogen atom, R4 represents a halogen atom, in particular a chlorine atom, and R5 represents a methyl group. In another particular embodiment, R6 represents a hydrogen atom, R4 represents a methyl group and R5 represents a halogen atom, in particular a chlorine atom.

According to one particular embodiment, R5 is a hydrogen atom and R4 and R6, which may be identical or different, represent a halogen atom, a C₁-C₆ alkyl group, a C₁-C₆ alkenyl group, or a C₁-C₆ alkyloxy group or a C₁-C₆ alkylthio group. According to another embodiment, R5 is a hydrogen atom and R4 and R6, which may be identical or different, represent a halogen atom, a C₁-C₆ alkyl group, a C₁-C₆ alkenyl group or a C₁-C₆ alkyloxy group. Even more particularly, R5 may be a hydrogen atom and R4 and R6, which may be identical or different, can represent a halogen atom, a C₁-C₆ alkyl group or a C₁-C₆ alkyloxy group. According to another particular embodiment, R5 is a hydrogen atom and R4 and R6, which may be identical or different, represent a halogen atom or a C₁-C₆ alkyl group. According to one preferred embodiment, R5 represents a hydrogen atom, R4 represents a halogen atom or a C₁-C₆ alkyl group, and R6 represents a halogen atom or a C₁-C₆ alkyl group. According to one implementation variant, R5 represents a hydrogen atom, R4 represents a halogen atom, and R6 represents a C₁-C₆ alkyl group. According to another variant, R5 represents a hydrogen atom, R4 represents a C₁-C₆ alkyl group, and R6 represents a halogen atom. According to yet another variant, R5 represents a hydrogen atom, R4 represents a C₁-C₆ alkyl group, in particular methyl, and R6 represents a C₁-C₆ alkyl group, in particular methyl. More particularly, R5 represents a hydrogen atom, R4 represents a halogen atom, in particular a chlorine atom, and R6 represents a methyl group. In another particular embodiment, R5 represents a hydrogen atom, R4 represents a methyl group and R6 represents a halogen atom, in particular a chlorine atom.

According to another embodiment, two groups from R1, R2 and R3 represent a hydrogen atom, and the other group from R1, R2 and R3 represents a C₃-C₆ alkyl group, a C₃-C₆ alkenyl group, C₃-C₆ alkyloxy group, a C₃-C₆ alkylthio group, a C₆-C₁₄ aryl group or a heteroaryl group comprising 5 to 14 ring members. More particularly, in another variant, the group from R1, R2 and R3 which is not a hydrogen atom is selected from a C₃-C₆ alkyl group, a C₆-C₁₄ aryl group and a heteroaryl group comprising 5 to 14 ring members. Even more particularly, the group from R1, R2 and R3 which is not a hydrogen atom is selected from a C₃-C₆ alkyl group and a C₆-C₁₄ aryl group. According to one particular embodiment, the C₃-C₆ alkyl group is a C₃-0₅ alkyl group. Preferentially, the two groups which represent hydrogens are R1 and R3. In this preferential embodiment, R2 can more particularly be selected from n-butyl, isobutyl or tert-butyl, more particularly n-butyl, groups and an aryl group, in particular phenyl.

According to another embodiment, two groups from R4, R5 and R6 represent a hydrogen atom, and the other group from R4, R5 and R6 represents a C₃-C₆ alkyl group, a C₃-C₆ alkenyl group, a C₃-C₆ alkyloxy group, a C₃-C₆ alkylthio group, a C₆-C₁₄ aryl group or a heteroaryl group comprising 5 to 14 ring members. More particularly, in another variant, the group from R4, R5 and R6 which is not a hydrogen atom is selected from a C₃-C₆ alkyl group, a C₆-C₁₄ aryl group and a heteroaryl group comprising 5 to 14 ring members. Even more particularly, the group from R4, R5 and R6 which is not a hydrogen atom is selected from a C₃-C₆ alkyl group and a C₆-C₁₄ aryl group. According to one particular embodiment, the C₃-C₆ alkyl group is a C₃-0₅ alkyl group. Preferentially, the two groups which represent hydrogens are R4 and R6. In this preferential embodiment, R5 can more particularly be selected from n-butyl, isobutyl, sec-butyl or tert-butyl, more particularly n-butyl, groups and an aryl group, in particular phenyl.

According to another particular embodiment, R1, R3, R4 and R6 represent a hydrogen atom. According to one implementation variant, R1, R3, R4 and R6 represent a hydrogen atom, and R2 and R5, which may be identical or different, preferably identical, represent a C₃-C₆ alkyl group, in particular an n-butyl, isobutyl, sec-butyl or tert-butyl, more particularly n-butyl, group or a C₆-C₁₄ aryl group, more particularly a C₆-C₈ aryl group. Preferentially, R2 and R5 are identical and represent an unsubstituted phenyl group, or a phenyl group substituted with one or more substituents selected from halogen atoms, C₁-C₆ alkyl groups, C₂-C₆ alkenyl groups, C₁-C₆ alkyloxy groups, C₁-C₆ alkylthio groups, C₂-C₆ ketone groups, C₁-C₆ ester groups, C₁-C₆ aldehyde groups, —NH₂, —CN, —CF₃, C₁-C₆ amine groups and C₁-C₆ amine groups mono- or disubstituted with a C₁-C₆ alkyl group, it being possible for the phenyl group to be more particularly substituted with one or more substituents selected from halogen atoms, C₁-C₆ alkyl group, C₂-C₆ alkene groups and alkyloxy groups. According to one preferred embodiment, R2 and R5 are identical and represent an unsubstituted phenyl group.

According to another embodiment, the R1 and R2 groups can form, with the carbon atoms to which they are bound, a ring comprising 5 to 7 ring members. Thus, mention may in particular be made of a ring forming, with the phenyl group to which it is bound, a naphthyl or benzothienyl group (embodiments illustrated by compounds P6 and P12). Likewise, the R4 and R5 groups can form, with the carbon atoms to which they are bound, a ring comprising 5 to 7 ring members, it being possible for this ring in particular to form, with the phenyl group to which it is bound, a naphthyl or benzothienyl group.

According to another advantageous embodiment, the compound of formula (I) is selected from the compounds of FIG. 1. According to one particularly preferred embodiment, the compound of formula (I) is selected from compounds P11, P9, P6, P15, P8, P12, P10 and P7 of FIG. 1. In one preferred embodiment, the compound of formula (I) is selected from 2-(dibiphenyl-4-ylboryloxy)ethamine (compound P11) and 2-(bis(4-(tert-butyl)phenyl)boryl)oxy)ethanamine (compound P9). Even more advantageously, the compound of formula (I) is compound P11.

The present invention also relates to pharmaceutically acceptable salts of the compounds of formula (I), and use thereof. In general, this term denotes the low-toxicity or non-toxic salts obtained from organic or inorganic bases or acids. These salts can be obtained during the final purification step for the compound according to the invention or by incorporation of the salt onto the compound already purified.

The compounds of formula (I) can be prepared according to methods known to those skilled in the art, who may in particular refer to the teachings of documents EP 1 444 981 and FR 3021050. The synthesis of compound P11 is in particular described in example 16 of document FR3021050. Other processes that are of use for the synthesis of the compounds of formula (I) have been described by Richard et al., Synthesis 2017, 49, pp. 736-744.

A compound of formula (I) can be formulated in the form of a pharmaceutical composition. The pharmaceutical compositions according to the invention advantageously comprise one or more pharmaceutically acceptable excipients or carriers. Mention may be made for example of buffered, isotonic, physiological, saline, etc., solutions compatible with pharmaceutical use and known to those skilled in the art. The compositions may contain one or more agents or carriers selected from dispersants, solubilizing agents, stabilizers, preservatives, etc. Agents or carriers that can be used in formulations (liquid and/or injectable and/or solid formulations) are in particular methylcellulose, hydroxymethylcellulose, carboxymethylcellulose, polysorbate 80, mannitol, gelatin, lactose, plant oils, acacia, liposomes, etc. The compositions can be formulated in the form of injectable suspensions, gels, oils, tablets, suppositories, powders, gel capsules, capsules, aerosols, etc., optionally by means of galenical forms and/or of devices providing sustained and/or delayed release. For formulations of this type, use is advantageously made of an agent such as cellulose, carbonates or starches. The compounds or compositions according to the invention can be administered in various ways and in various forms. Thus, they can be for example administered systemically, orally, parenterally, by inhalation or by injection, for instance intravenously, intramuscularly, subcutaneously, transdermally, intra-arterially, etc. For injections, the compounds are generally conditioned in the form of liquid suspensions, which can be injected by means of syringes or drips, for example.

It is demonstrated herein that the compounds of formula (I) are capable of inhibiting SOCE with a submicromolar Ki, and that this inhibitory activity is selective for Orai1-SOCE at low dose, in comparison to its effect against IP3R and SERCA pumps. The compounds of formula (I), more particularly compounds P9 and P11, are therefore particularly useful for inhibiting Orai1-SOCE. An object of the invention is therefore a compound of formula (I), for use in the inhibition of Orai1-SOCE. An object of the invention is therefore also a compound of formula (I) for use in the modulation of calcium channels in an immune cell.

More generally, an object of the invention is a compound of formula (I), for use as a medicament, in particular as an immunosuppressant. Under these conditions, the compounds of formula (I) can thus in particular be used in order to prevent graft rejection.

An object of the invention is also a compound of formula (I) for the treatment of pulmonary arterial hypertension.

The invention is directed more particularly toward a compound of formula (I) for use as an inhibitor of immune cells, in particular leukocytes such as T lymphocytes and B lymphocytes, as well as monocytes.

The invention also relates to a compound of formula (I) for use in the inhibition of immune cell functions. By way of illustration, mention may be made of the use thereof for inhibiting the production of cytokines, in particular of cytokines which regulate immune cell activation, more particularly for inhibiting IL-2 production by T lymphocytes. Mention may also be made of the use thereof for inhibiting the production of oxygen free radicals by neutrophils or for inhibiting phagocytosis by macrophages (functions involved in chronic inflammations).

The compounds of formula (I) can be used for the treatment of a disorder for which the use of an Orai1-SOCE inhibitor is indicated. The term “treatment” denotes curative, symptomatic or preventive treatment. The compounds of the present invention can thus be used in subjects (such as mammals, in particular humans) suffering from a declared disease. The compounds of the present invention can also be used for delaying or slowing down the progression of the disease, or preventing further progression of the disease, thus improving the condition of the subjects. The compounds of the present invention can finally be administered to subjects who are not ill, but who could normally develop the disease or who have a high risk of developing the disease.

In this respect, the invention is directed in particular toward a compound of formula (I), for use thereof in a method for treating an inflammatory disorder, in particular a chronic or acute, more particularly chronic, inflammatory disorder.

The invention also relates to a compound of formula (I) for use in the treatment of an allergic disorder.

The invention is also directed toward a compound of formula (I) for use in a method for treating an immune disorder. Among the immune disorders that can be treated thanks to the compound of formula (I), mention may in particular be made of autoimmune diseases and inflammatory diseases, more particularly chronic inflammatory diseases. By way of illustration, the compound of formula (I) can be used in the treatment of chronic inflammatory diseases such as rheumatoid arthritis, pancreatitis, bowel inflammations, such as Crohn's disease or proctitis, psoriasis or type 2 diabetes. Moreover, the autoimmune diseases that can be treated with the compound of formula (I) correspond to all diseases involving an excessive action of the immune system against a cell, a tissue, or an organ of the subject to be treated. By way of illustration, mention may be made of psoriasis, lupus, vasculitis, rheumatoid arthritis, multiple sclerosis, vitiligo, scleroderma, type 1 diabetes or autoimmune hepatitis.

The invention also relates to a compound of formula (I), for use in the treatment of a cancer. The invention is directed more particularly toward the treatment of a cancer involving Orai1. More particularly, the invention is directed toward a compound of formula (I) for use in the treatment of a blood cancer, in particular of leukemia or of lymphoma, or of breast cancer. According to another particular embodiment, the invention is directed toward a compound of formula (I) for use in the treatment of a hormone-independent cancer, in particular hormone-independent breast cancer. In the context of the present invention, a cancer is described as “hormone-independent cancer” if female hormones (in particular estrogens or progesterone) do not play a role in the proliferation of the cancer cells. Those skilled in the art are able to determine whether a cancer is hormone-independent, in particular by anatomical-pathological examination.

Moreover, the invention relates to a compound of formula (I), for use in the treatment of diseases termed “gain-of-function diseases”, such as Stormorken syndrome or tubular aggregate myopathy caused by mutations of the Orai1 gene resulting in overactivity of the Orai1 channels and constitutive SOCE.

The invention also relates to a method for treating a disorder for which the use of an Orai1-SOCE inhibitor is indicated, more particularly one of the disorders mentioned above, comprising the administration of an effective amount of a compound of formula (I). For the purposes of the invention, the term “an effective amount” refers to an amount of the compound that is sufficient to produce the desired biological result. For the purposes of the present invention, the term “subject” signifies a mammal and more particularly a human being. The amount of compound of formula (I) to be administered can vary to a large extent, and will depend in particular on the disorder to be treated and on the stage thereof, on the age, sex or weight of the subject to be treated, on the route of administration and/or on the duration of the treatment. In the case of a human subject, the orally administered dose can in particular be between 1 mg and 4000 mg, in particular between 1 mg and 1000 mg, one or more times a day. Parenteral administration, in particular intravascular administration, more particularly intravenous or intra-arterial administration, preferentially intravenous administration, may in particular be carried out at a dose of between 0.1 and 100 mg, one or more times a day, or administration may be continuous for 1 to 24 hours, intravenously.

The following examples illustrate the invention, without however limiting it.

### FIGURE LEGENDS

FIG. 1 represents the structure of the 2-APB compound (compound P1) and of the 2-APB analogs disclosed in the present application.

FIG. 2 shows that 2-APB has a dual effect on the SOCE of Jurkat T cells, whereas the other 2-APB analogs have only an inhibitory effect.

FIG. 3 is a graph showing that compound P11 inhibits Mn²⁺ influx via the SOCCs and SOCE of other cell types.

FIG. 4 is a graph showing that compounds P9 and P11 preferentially inhibit SOCE compared to loading via SERCA and compared to calcium release from the ER by IP3R (“IICR”).

FIG. 5 is a graph showing that compound P11 increases cell death and decreases interleukin-2 synthesis by PHA-stimulated Jurkat cells.

FIG. 6 is a graph showing that compound P11 increases the caspase-3 activity of PHA-stimulated Jurkat cells.

FIG. 7 is a graph showing that compound P11 increases DNA fragmentation in PHA-stimulated Jurkat cells.

### EXAMPLES

1. Materials and Methods

The Jurkat T lymphocyte line was mainly used for characterizing the effect of the molecules on SOCE. The Indo-1 ratiometric probe was used to visualize cytosolic calcium variations. For this, the cells in culture were centrifuged and then resuspended and incubated in PBS (Phosphate Buffered Saline) medium supplemented with 1 mg/ml of albumin and 4 μM of Indo-1-AM for 45 mim at ambient temperature and in the dark. The cells were then again centrifuged and resuspended in the HBS (Hepes-Buffered Saline) recording medium having the composition (in mM): 135 mM NaCl, 5.9 mM KCl, 1.2 mM MgCl₂, 11.6 mM Hepes, 11.5 mM glucose, pH adjusted to 7.3 with NaOH. 0.5 to 1 million cells are then placed in the cuvette of a Varian Cary Eclipse spectrofluorimeter and the Indo-1 probe is excited at 360 nm, with emission of fluorescence at 405 and 480 nm. The ratio of fluorescence at 405 and 480 nm is directly proportional to the [Ca²⁺]cyt.

The lines of monocyte origin U937, B lymphocyte origin (DG75) and breast cancer origin (MDA-MB231) were also used with the same protocol.

To test the selectivity of certain molecules on SOCE compared to IP3R and SERCA, the L15 line stably expressing type 1 IP3R was used. Briefly, the cells were permeabilized with saponin and incubated in the presence of ⁴⁵Ca²⁺ in a medium containing ATP allowing SERCA activity and Ca' ion entry into the ER. The addition of TG makes it possible to induce release from the ER and therefore to measure the amount of Ca²⁺ ions that have left the ER, which is itself directly linked to the SERCA activity. The addition of IP3 allows direct measurement of the amount of Ca' ions leaving the ER by IP3R opening.

In order to measure the interleukin-2 (IL-2) synthesis in 24 h after stimulation with 10 μg/ml of phytohemagglutinin (PHA) in the presence or absence of certain molecules, the Jurkat cells are centrifuged and the supernatant containing the IL-2 is collected. The 11-2 concentration is measured by Elisa (R&D systems kit).

The caspase-3 activity is measured with a fluorescent kit using the Ac-DEVD-AFC substrate. The Jurkat cells are incubated for 24 h with PHA in the presence or absence of certain molecules. The cells are then lyzed with RIPA medium, and the amounts of proteins are then measured with a commercial kit (Biorad). The cell lysates are then diluted in the following reaction medium: 200 mM Hepes, pH 7.4, 1 mM EDTA, 20% sucrose and 20 mM dithiothreitol, then incubated with 20 μM of the fluorogenic substrate for 45 min at 7 h at 37° C. The appearance of the fluorescent AFC product is measured by spectrofluorimetry (excitation at 405 nm, emission at 505 nm).

The Roche TUNEL kit was used to observe the nuclear DNA fragmentation and therefore cell apoptosis.

2. Results

2.1. Effects of the Analogs on Cytosolic Calcium Concentration

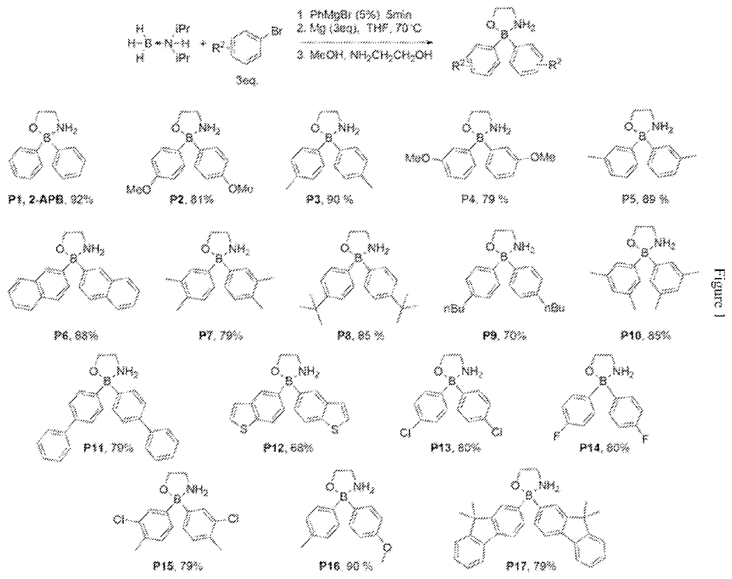

New analogs of 2-APB were synthesized in order to identify strong SOCE inhibitors. The molecules tested and their synthesis route are presented in FIG. 1.

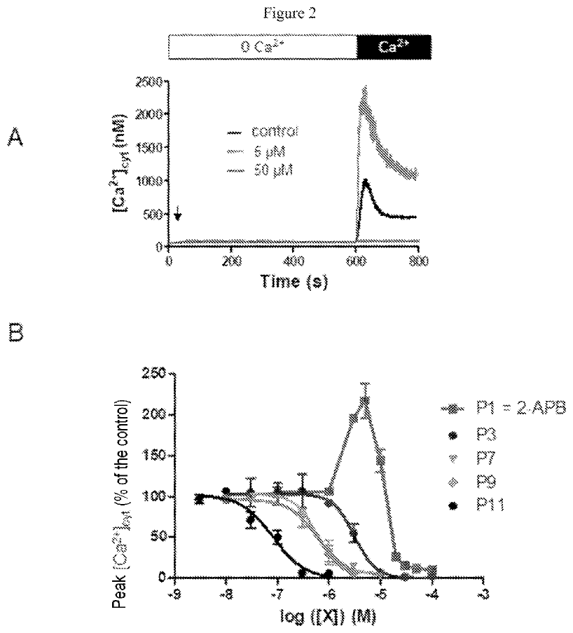

The SOCE phenomenon was evaluated conventionally. Jurkat cells were placed in calcium-free HBS medium and stimulated with 1 μM of thapsigargin (TG, arrow in FIG. 2A) for 600 s in order to allow calcium release from the ER and SOCC channel opening(=CRAC). The addition of 1 mM of CaCl₂ to the medium allows massive calcium ion entry through the SOCCs and an increase in the concentration of cytosolic calcium ([Ca²⁺]_{cyt}).

The synthesis of the new compounds was validated by evaluating the effect of 2-APB synthesized under the same conditions. FIG. 2A shows that 2-APB synthesized in this way clearly has its conventional properties: high potentiation of calcium entry at 5 μM and virtually total inhibition at 50 μM.

The dose-response curves were established after addition of 2-APB or of an analog thereof 30 seconds before the reintroduction of calcium. The results show that the 2-APB analogs do not exhibit the property of increasing cytosolic calcium concentration. The increase in the size of the molecules at the level of the phenyl groups therefore has a negative effect on the potentiation activity. The analogs tested are therefore SOCE inhibitors without the effect of potentiation of the increase in cytosolic calcium normally observed at low dose with 2-APB (cf. table 1).

TABLE 1

| Compound | Ki (nM) |
|---|---|
| P11 | 75 ± 21 |
| P6 | 275 ± 17 |
| P15 | 294 ± 74 |
| P8 | 350 ± 40 |
| P12 | 374 ± 96 |
| Dibenzothienyl-APB | 405 ± 23 |
| (Djillani 2014) | |
| P10 | 484 ± 116 |
| P9 | 641 ± 103 |
| P7 | 751 ± 97 |
| Compound | Ki (μM) |
| P13 | 1.84 ± 0.1 |
| P5 | 2.1 ± 0.6 |
| P16 | 3.1 ± 0.6 |
| P3 | 3.5 ± 0.6 |
| P2 | 3.5 ± 0.65 |
| P14 | 5.4 ± 0.3 |
| P4 | 75 ± 21 |
| P17 | >>1 μM |

Compounds P3 and P5 comprise a methyl group on each of the phenyl groups of the molecule (in the para and meta positions, respectively). P7 and P10 correspond to compounds P3 and P5 to which a second methyl group has been added on each of the phenyl groups (a group in the meta-position and a group in the para-position of each of the phenyls). These modifications result in a significant increase in the efficiency of inhibition of the molecules. However, compound P10 is the most active of the two, with a Ki of approximately 500 nM and a total inhibition obtained at 3 μM.

Other analogs were generated with a methoxy group on each of the phenyl groups of the 2-APB (compounds P2 and P4). It is observed that the placement of the methoxy group in the para-position makes it possible to obtain a compound (compound P2) having an activity equivalent to that of the compound comprising a methyl group at this position (compound P3). On the other hand, the placement of this methoxy group in the meta-position (compound P4) has a drastic negative impact on the inhibition constant, which rises to 75±21 μM, with a maximum inhibition of 80% only. Compound P16 combines a methyl group in the para-position of one of the phenyls, and a methoxy group on the other phenyl. This compound has an inhibitory activity similar to compounds P2 and P3 (3.1±0.6 μM).

Halogenated analogs were also prepared. These are compounds P13, P14 and P15. Compounds P13 and P14, wherein a chlorine and a fluorine are located in the para-position of each of the phenyl groups, respectively, have an activity equivalent to compound P3. Surprisingly, compound P15, which is similar to compound P7 in which a chlorine atom would have been replaced with methyl groups in the meta-position of each of the phenyl groups, has an improved inhibitory activity with a Ki of approximately 300 nM.

Compounds P8 and P9 are analogs comprising, in the para-position of the phenyl groups, substituents that are bulkier than the methyl group of compound P3, with respectively a tert-butyl and n-butyl group. This modification leads to a significant increase in the inhibitory activity of the analogs, with a Ki of 350±40 nM and 641±103 nM, respectively. It is therefore demonstrated that the addition of bulkier groups leads to an increase in the inhibitory activity of the compounds.

Moreover, analogs comprising a naphthyl group (compound P6) or benzothienyl group (compound P12) in place of each phenyl group of 2-APB were synthesized. Compounds P6 and P12 exhibit a significant inhibitory activity, with a Ki of 275±17 nM and 374±96 nM, respectively.

However, the best inhibitor identified is compound P11, which comprises a phenyl group bonded in the para-position of each of the phenyl groups of 2-APB, and has a Ki of 75±21 nM on Jurkat cells. Entirely surprisingly, the imposition of a constraint between the two phenyl groups by means of the introduction of a gem-dimethyl (compound P17) leads to a total absence of activity of the compound, resulting in a Ki of greater than 3 μM. Free rotation of the two phenyl groups of compound P11 therefore appears to be an important parameter for maintaining the very high efficiency of said compound.

2.2. Specific Inhibition of SOCE without Impact on Calcium Efflux Mechanisms

In order to determine whether compound P11 has an action on SOCE and not on calcium efflux mechanisms, a quenching experiment with Mn²⁺ was carried out by means of the indo-1 molecule. The Mn²⁺ ions enter the cells via the SOCCs, but cannot be pumped into the extracellular membrane by the plasma membrane calcium-dependent ATPases (plasma membrane Ca²⁺ ATPases, or PMCA) or the sodium/calcium exchanger (Na⁺/Ca²⁺ exchanger, or NCX) or sent back into the lumen of the ER by the sarcoplasmic-endoplasmic reticulum Ca²⁺ ATPases (or SERCAs). Once in the cell, Mn²⁺ binds to indo-1 and quenches its fluorescence measured at 430 nm. An increase in the amplitude of the Mn²⁺ influx is therefore associated with an increase in the indo-1 quenching rate, and vice versa.

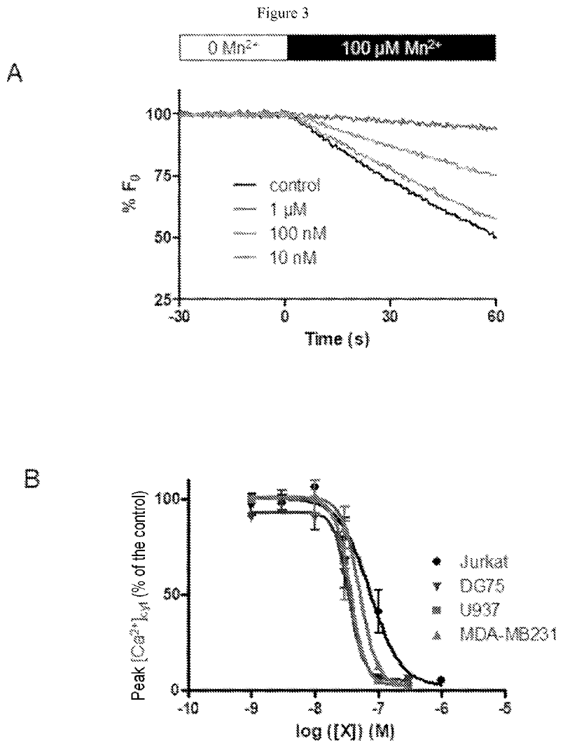

After 10 minutes of treatment with TG in order to open the SOCCs, 100 μM of MnCl₂ were added. A degree of quenching of −0.89±0.05% Fo/s was observed (FIG. 3A). In the presence of increasing concentrations of P11 added 30 seconds before the addition of Mn²⁺ ions, the degree of quenching decreased and reached a blocking of approximately 90% at 1 μM (−0.09±0.01, FIG. 3A).

Compound P11 therefore clearly targets SOCE.

Moreover, FIG. 3B shows that P11 is also capable of blocking SOCE in other cell types, with an efficiency significantly greater than that observed in Jurkat cells. Thus, Ki values of 32±2 nM, 40±5 nM and 50±5 nM were calculated in DG75 cells (B lymphocytes), U937 cells (monocytes) and MDA-MB231 cells (hormone-independent breast cancer cells) respectively, compared with 75±21 nM for Jurkat cells (FIG. 3B). FIG. 3B also shows that total inhibition is achieved at 100 nM.

2.3. Selectivity of the Compounds of the Invention

The selectivity of compound P11 and of the close compound P9 was evaluated.

The ⁴⁵Ca²⁺ loading and IP3-dependent release measurements were carried out on permeabilized cells under one-way conditions.

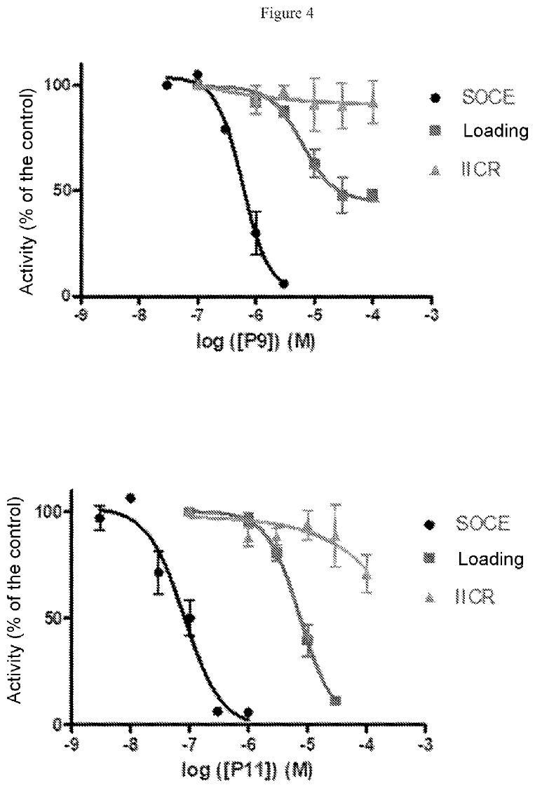

FIG. 4 shows the results obtained with P9 and P11.

P9 did not significantly inhibit the activity of IP3R and is only a partial inhibitor of SERCA activity, with an inhibition of −52±3% at 100 μM and a Ki of 6±0.3 μM. Below 3 μM, compound P9 inhibits only SOCE.

P11 inhibits IP3R only very slightly, with a value of 29±9% at 100 μM, the calculation of the Ki with respect to this receptor having moreover proved to be impossible because of the very high concentrations that would have been required in order to obtain this value. SERCA is 89% inhibited by P11, at a concentration greater than 30 μM. However, this activity is quite unlike that observed on SOCE, since the Ki for SERCA is 7.4 μM, whereas that for SOCE is 75 nM, that is to say 100 times less for the latter. As a result of this, P11 is a more selective inhibitor of SOCE at concentrations of less than 1 μM, without any effect on SERCA and IP3R.

2.4. The Compounds of the Invention Prevent Jurkat Cell Activation

T lymphocyte activation requires an increase in cytosolic calcium concentration for several minutes in order to allow interleukin 2 (IL-2) synthesis and immune function activation. Consequently, inhibition of the increase in cytosolic calcium concentration due to SOCE prevents the activation of T lymphocytes and proliferation thereof.

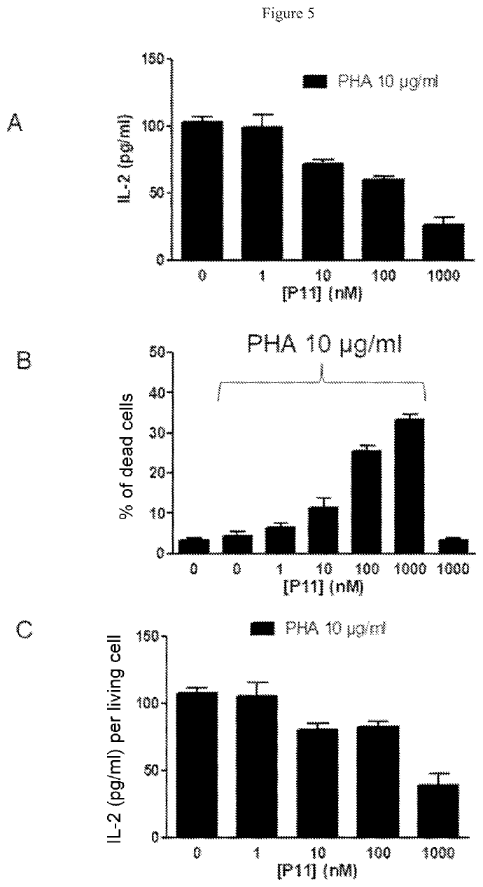

The concentration of neosynthesized IL-2 was measured in the culture medium of phytohemagglutinin (PHA)-stimulated Jurkat cells after 24 h, in the presence of increasing concentrations of compound P11. In the absence of stimulation by PHA, Jurkat cells do not produce IL-2. Conversely, stimulation with PHA allows the detection of approximately 100 pg/ml of IL-2 (103 ±4 pg/ml, FIG. 5A). The addition of increasing concentrations of P11 at the same time as the stimulation by PHA leads to a gradual decrease in IL-2 synthesis to 26±6 pg/ml for 1 μM of P11 (-74%, FIG. 5B).

PHA alone or P11 alone did not exhibit any toxic effect on the cells. However, the combination of the treatment of the cells with PHA and increasing concentrations of P11 significantly increases the percentage of dead cells (which goes from approximately 5% without treatment, to 33.2±1.3% at 1 μM of P11, FIG. 5B). However, the calculation of the amount of IL-2 synthesized by the remaining living cells shows that increasing amounts of P11 decrease the capacity of synthesis of this cytokine by the T lymphocytes by approximately 63%, going from 107.6±4.1 pg/ml to 39.2±8.6 pg/ml (FIG. 5C).

P11 therefore has a double effect on activated Jurkat cells: a reduction in IL-2 synthesis and an induction of cell death.

2.5. Induction of Apoptosis of the Activated Jurkat Cells

The caspase-3 activity was then evaluated by means of an Ac-DEVD-AFC fluorogenic substrate, and apoptosis was evaluated by means of a TUNEL assay.

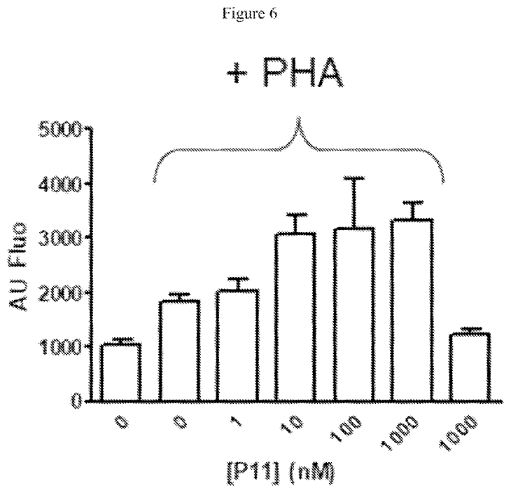

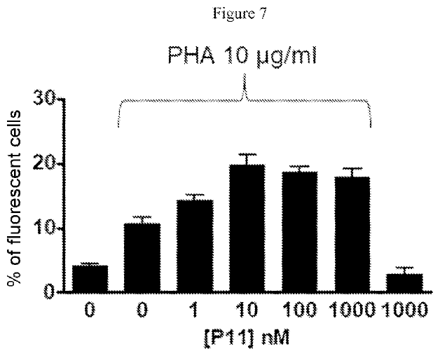

FIG. 6 shows that compound P11 alone does not induce an increase in caspase-3 activity, including at a concentration of 1 μM. The caspase-3 activity increases by a factor of 2 after stimulation with PHA for 24 h (1836±132 arbitrary units (AU) compared with 1037±100 AU). The supplementary addition of P11, this being from 10 nM, leads to a maximum activation effect with an increase by a factor of 3 (3332±113 AU at 1 μM). Similar results were obtained by means of the TUNEL assay, which shows that, at concentrations greater than 10 nM, compound P11 induces a 4.5-fold increase in the number of cells containing fragmented DNA (17.9±1.2%), in cells stimulated with PHA (FIG. 7).

These results therefore show that compound P11 does not exhibit a toxic effect on the non-activated cells, but that it has an impact on Jurkat cell activation through an induction of apoptosis of said cells.

1-11. (canceled)

12. A method for inducing immunosuppression in a subject in need thereof, comprising administering to said subject a compound of formula (I) or a pharmaceutically acceptable salt thereof: 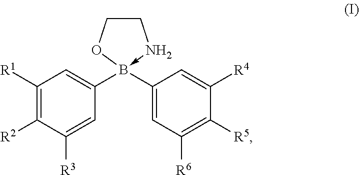
wherein R1, R2, R3, R4, R5 and R6 are groups, which may be identical or different, selected from a hydrogen atom, a halogen atom, a C₁-C₆ alkyl group, a C₁-C₆ alkenyl group, a C₁-C₆ alkyloxy group, a C₁-C₆ alkylthio group, a C₆-C₁₄ aryl group and a heteroaryl group comprising 5 to 14 ring members;
with the proviso that, when two of the R1, R2 and R3 groups represent a hydrogen, then the group from R1, R2 and R3 which is not a hydrogen is selected from a C₃-C₆ alkyl group, a C₃-C₆ alkenyl group, a C₃-C₆ alkyloxy group, a C₃-C₆ alkylthio group, a C₆-C₁₄ aryl group and a heteroaryl group comprising 5 to 14 ring members;
with the proviso that, when two of the R4, R5 and R6 groups represent a hydrogen, then the group from R4, R5 and R6 which is not a hydrogen is selected from a C₃-C₆ alkyl group, a C₃-C₆ alkenyl group, a C₃-C₆ alkyloxy group, a C₃-C₆ alkylthio group, a C₆-C₁₄ aryl group and a heteroaryl group comprising 5 to 14 ring members;
it being possible for the R1 and R2 groups to form, with the carbon atoms to which they are bound, a ring comprising 5 to 7 ring members;
it being possible for the R4 and R5 groups to form, with the carbon atoms to which they are bound, a ring comprising 5 to 7 ring members; and
said aryl or heteroaryl groups being optionally substituted with one or more substituents selected from halogen atoms, C₁-C₆ alkyl groups, C₂-C₆ alkenyl groups, C₁-C₆ alkyloxy groups and C₁-C₆ alkylthio groups, C₂-C₆ ketone groups, C₁-C₆ ester groups, C₁-C₆ aldehyde groups, —NH₂, −CN, —CF₃, C₁-C₆ amine groups and C₁-C₆ amine groups mono- or disubstituted with a C₁-C₆ alkyl group.

13. The method according to claim 12, wherein R1, R3, R4 and R6 represent a hydrogen atom and R2 and R5, which are identical, represent a C₃-C₆ alkyl group, in particular an n-butyl, isobutyl, sec-butyl or tert-butyl, more particularly n-butyl, group, or a C₆-C₁₄ aryl group, more particularly a C₆-C₈ aryl group.

14. The method according to claim 13, wherein R2 and R5 represent a substituted or unsubstituted phenyl group.

15. The method according to claim 12, said compound being 2-(dibiphenyl-4-ylboryloxy)ethamine (compound P11).

16. The method according to claim 12, said compound being 2-(bis(4-(tert-butyl)phenyl)boryl)oxy)ethamine (compound P9).

17. The method according to claim 12, wherein:
R3 is a hydrogen atom and R1 and R2, which may be identical or different, represent a halogen atom or a C₁-C₆ alkyl group; and/or
R6 is a hydrogen atom and R4 and R5, which may be identical or different, represent a halogen atom or a C₁-C₆ alkyl group.

18. The method according to claim 12, wherein:
R2 represents a hydrogen atom, and R1 and R3, which may be identical or different, represent a halogen atom or a C₁-C₆ alkyl group; and/or
R5 is a hydrogen atom and R4 and R6, which may be identical or different, represent a halogen atom or a C₁-C₆ alkyl group.

19. The method according to claim 12, for inhibiting cytokine production by an immune cell, in particular for inhibiting IL-2 production from T lymphocytes.

20. A method for treating an inflammatory disorder, an immune disorder, an allergy or a cancer, in a subject in need thereof, comprising administering to said subject a compound of formula (I) or a pharmaceutically acceptable salt thereof: 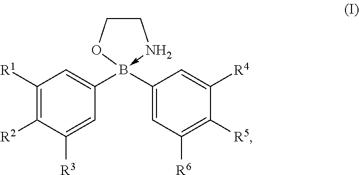
wherein R1, R2, R3, R4, R5 and R6 are groups, which may be identical or different, selected from a hydrogen atom, a halogen atom, a C₁-C₆ alkyl group, a C₁-C₆ alkenyl group, a C₁-C₆ alkyloxy group, a C₁-C₆ alkylthio group, a C₆-C₁₄ aryl group and a heteroaryl group comprising 5 to 14 ring members;
with the proviso that, when two of the R1, R2 and R3 groups represent a hydrogen, then the group from R1, R2 and R3 which is not a hydrogen is selected from a C₃-C₆ alkyl group, a C₃-C₆ alkenyl group, a C₃-C₆ alkyloxy group, a C₃-C₆ alkylthio group, a C₆-C₁₄ aryl group and a heteroaryl group comprising 5 to 14 ring members;
with the proviso that, when two of the R4, R5 and R6 groups represent a hydrogen, then the group from R4, R5 and R6 which is not a hydrogen is selected from a C₃-C₆ alkyl group, a C₃-C₆ alkenyl group, a C₃-C₆ alkyloxy group, a C₃-C₆ alkylthio group, a C₆-C₁₄ aryl group and a heteroaryl group comprising 5 to 14 ring members;
it being possible for the R1 and R2 groups to form, with the carbon atoms to which they are bound, a ring comprising 5 to 7 ring members;
it being possible for the R4 and R5 groups to form, with the carbon atoms to which they are bound, a ring comprising 5 to 7 ring members; and
said aryl or heteroaryl groups being optionally substituted with one or more substituents selected from halogen atoms, C₁-C₆ alkyl groups, C₂-C₆ alkenyl groups, C₁-C₆ alkyloxy groups and C₁-C₆ alkylthio groups, C₂-C₆ ketone groups, C₁-C₆ ester groups, C₁-C₆ aldehyde groups, —NH₂, —CN, —CF₃, C₁-C₆ amine groups and C₁-C₆ amine groups mono- or disubstituted with a C₁-C₆ alkyl group.

21. The method according to claim 20, wherein R1, R3, R4 and R6 represent a hydrogen atom and R2 and R5, which are identical, represent a C₃-C₆ alkyl group, in particular an n-butyl, isobutyl, sec-butyl or tert-butyl, more particularly n-butyl, group, or a C₆-C₁₄ aryl group, more particularly a C₆-C₈ aryl group.

22. The method according to claim 21, wherein R2 and R5 represent a substituted or unsubstituted phenyl group.

23. The method according to claim 20, said compound being 2-(dibiphenyl-4-ylboryloxy)ethamine (compound P11).

24. The method according to claim 20, said compound being 2-(bis(4-(tert-butyl)phenyl)boryl)oxy)ethamine (compound P9).

25. The method according to claim 20, wherein:
R3 is a hydrogen atom and R1 and R2, which may be identical or different, represent a halogen atom or a C₁-C₆ alkyl group; and/or
R6 is a hydrogen atom and R4 and R5, which may be identical or different, represent a halogen atom or a C₁-C₆ alkyl group.

26. The method according to claim 20, wherein:
R2 represents a hydrogen atom, and R1 and R3, which may be identical or different, represent a halogen atom or a C₁-C₆ alkyl group; and/or
R5 is a hydrogen atom and R4 and R6, which may be identical or different, represent a halogen atom or a C₁-C₆ alkyl group.

27. The method according to claim 20, for treating breast cancer in a subject in need thereof.

28. The method according to claim 20, for treating a hormone-independent cancer.

29. The method according to claim 20, for the treating a chronic inflammatory disorder, leukemia, lymphoma, pulmonary arterial hypertension or graft rejection.
